(19) United States
(12) Patent Application Publication
(10) Pub. No.: US 2005/0208163 A1
(43) Pub. Date: Sep. 22, 2005

(54) Compositions containing avocado leaf extract for lowering cholesterol levels

(76) Inventors: Ernesto A. Brovelli, Corona, CA (US); Julio Andres Vallejos, Corrientes (AR); Chioma Ikonte, West Covina, CA (US); Gopi R. Menon, Riverside, CA (US); David J. Fast, Grand Rapids, MI (US); Amitabh Chandra, Grand Rapids, MI (US)

Correspondence Address: ALTICOR INC., 7575 FULTON STREET EAST MAILCODE 78-2G, ADA, MI, 49355, US

(21) Appl. No.: 10/855,301

(22) Filed: May 27, 2004

Related U.S. Application Data
(63) Continuation-in-part of application No. 10/209,021, filed on Jul. 31, 2002, now Pat. No. 6,793,946.

(51) Int. Cl. A61K 35/78

(52) U.S. Cl. 424/769

(57) ABSTRACT

The present invention relates to compositions containing Persea american var. drymifolia leaf and leaf extracts for lowering cholesterol levels and methods thereof.

### RELATED APPLICATIONS

This patent application is a continuation-in-part of application Ser. No. 10/209,021 filed Jul. 31, 2002 which is incorporated in its entirety by reference.

### FIELD OF THE INVENTION

The present invention relates to compositions containing avocado leaf for lowering cholesterol levels in mammals. More particularly, the present invention relates to compositions containing an extract from avocado leaf of the species Persea americana var. drymifolia (hereinafter referred to as “drymifolia”) for use in lowering cholesterol levels in humans.

### BACKGROUND OF THE INVENTION

Cardiovascular disease (CVD) remains the leading cause of illness and death in at least North America. www.americanheart.org, American Heart Association, 1999 Heart and Stroke Statistical Update. A major risk factor for CVD is elevated blood cholesterol levels. Cholesterol is a soft, waxy substance found among the lipids in the bloodstream and in the cells of the human body. Although cholesterol serves needed bodily functions, high cholesterol levels in the blood may be detrimental to a person's health because it increases the risk of CVD.

High cholesterol generally means that a person's total blood cholesterol level is more than 240 mg/dl or that a person's low density lipoprotein level is more than 160 mg/dl. Cleeman, James I., “Executive Summary of the Third Report of the National Cholesterol Education Program (NCEP) Expert Panel on Detection, Evaluation, and Treatment of High Blood Cholesterol in Adults (Adult Treatment Panel III),” JAMA 285(19): 2486-2497 (2001). Approximately 41.3 million Americans have total blood cholesterol levels of 240 mg/dL or higher. www.americanheart.org, American Heart Association, Biostatistical Fact Sheet, (2002). High total cholesterol levels are primarily related to elevated levels of low density lipoproteins (LDL) which are the major cholesterol carrier in the blood stream. When LDL is elevated, it can build up on artery walls. This condition, called atherosclerosis, increases the risk of blood clots, heart attack, and stroke. High density lipoproteins (HDL), on the other hand, carry cholesterol away from the arteries and is, thus, considered “good cholesterol.”

Because of the known link between high total cholesterol and CVD, there remains a considerable amount of interest in regulating cholesterol levels in the body. One common intervention is a class of lipid-regulating pharmaceuticals called statins. A popular statin is LIPITOR®. Although these agents have been proven safe in clinical trials, like any drug, they carry the risk for undesirable side-effects.

61 It has now been found that drymifolia leaves contain agents that lower total cholesterol levels. Accordingly, compositions containing dymifolia leaf and dymifolia leaf extracts are believed to be natural alternatives to LIPITOR® and other prescription drugs.

### SUMMARY OF THE INVENTION

The present invention relates to a cholesterol lowering composition containing drymifolia leaf and drymifolia leaf extracts. In one embodiment the drymifolia leaves are dehydrated, milled and prepared for consumption as a tea. Surprisingly, the leaves of drymifolia showed a cholesterol lowering effect comparable to LIPITOR® and a much greater cholesterol lowering effect than the fruit and the leaves of other avocado varieties commonly grown in North America, namely, Persea nubigena var. guatamalensis cv. Nabal and Persea nubigena var. guatamalensis cv. Haas. In another embodiment, compositions, such as dietary supplements, are provided containing an extract prepared from dehydrated drymifolia leaves. In a third embodiment, a composition comprising drymifolia leaf extract in combination with other cholesterol lowering agents is provided. In a fourth embodiment a method of lowering cholesterol levels in a mammal by administering a drymifolia leaf extract is provided. These and other aspects and advantages of the present invention will be better understood by reference to the drawings and the detailed description of the preferred embodiment.

### BRIEF DESCRIPTION OF THE DRAWINGS

FIG. 1 is a graph displaying dose specific responses of Hep G2 cells to hand-crushed and milled leaves of drymifolia in comparison to LIPITOR® in cholesterol release.

FIG. 2 is a graph displaying the dose specific responses of Hep G2 cells to milled leaves of drymifolia in comparison to milled leaves of Persea nubigena var. guatamalensis cv. Nabal and milled leaves of Persea nubigena var. guatamalensis cv. Haas in cholesterol release.

FIG. 3 is a graph displaying dose specific responses of Hep G2 cells to milled leaves of drymifolia in comparison to the fruit of Persea nubigena var. guatamalensis cv. Haas in cholesterol release.

FIG. 4 is a graph displaying dose specific responses of Hep G2 cells to milled leaves of drymifolia in comparison to drymifolia leaf extract in cholesterol release. The data are expressed as % control cholesterol from untreated cells.

FIG. 5 is a graph displaying dose specific responses of Hep G2 cells to pure rutin, LIPITOR®, and four drymifolia leaf extract samples in cholesterol release.

FIG. 6 is a bar graph showing the responses of Hep G2 cells to drymifolia leaf extract in combination with other cholesterol lowering agents in cholesterol release.

### DETAILED DESCRIPTION OF THE PREFERRED EMBODIMENT

The present invention comprises compositions having drymifolia leaves and drymifolia leaf extracts, both from Persea americana var. drymifolia (hereinafter referred to as “drymifolia”) for lowering cholesterol levels in mammals. This variety is available in Northern Argentina and Mexico and is known to have an anise scent. The particular substance(s) in drymifolia leaves that causes cholesterol lowering is not yet understood. However, the leaves prepared as a tea and extracts derived from the leaves show cholesterol lowering activity.

It is a commonly understood principle in the herbal industry that active ingredients in fresh plants/herbs generally decompose or diminish in effectiveness as the plant/herb dies or decays. Thus, to preserve the cholesterol reducing capacity of drymifolia leaves for shipment, fresh drymifolia leaves can be air dried at average room temperatures of about 16° C. (about 60° F.) to about 27° C. (about 80° F.). Surprisingly, drying at room temperature maintains the efficacy of the drymifolia leaves in lowering cholesterol. The drymifolia leaves can also be oven dried at about 80° C. (about 176° F.). The drying temperature is maintained until the leaves are prepared for consumption. Once dehydrated, the leaves are hand-crushed and, more preferably, are milled. Hand-crushed leaves showed better cholesterol lowering activity than the fruit and leaves of Persea nubigena var. guatamalensis cv. Nabal (“Nabal”) and the leaves of Persea nubigena var. guatamalensis cv. Haas (“Haas”), where milled drymifolia leaves showed greater cholesterol reduction than hand-crushed drymifolia leaves. Milling can be performed by a Glen Mills hammer mill bench top unit (Mot. KM 80-60, Culatti Typ MFC). Milled and dehydrated drymifolia leaves can be used to prepare a tea or further processed into an extract for use in, for example, a dietary supplement, beverage or food.

Tea

To prepare a tea in accordance with one embodiment of the present invention, dehydrated drymifolia leaves are milled using a 2 mm screen to obtain an average particle size of about 28 microns. More preferably, the leaves may be milled twice using a 1 mm screen to provide an average particle size of about 9 microns. Most preferably, the dehydrated leaves may be milled using a 1 mm screen, providing an average particle size of about 11 microns. The tea is prepared as a 1% extract by preparing 10 mg of hand-crushed drymifolia leaves or milled drymifolia leaves per 1 ml of water. In one embodiment, 100 mg of drymifolia leaves are placed in 10 ml of water to form a tea. The tea is boiled for about 1 minute to about 10 minutes. More preferably, the tea is boiled for about 3 minutes to about 7 minutes and, most preferably, for about 5 minutes. The water can be boiling after or upon immersing the drymifolia leaves in the water. It will be appreciated by those of ordinary skill in the art that the boiling times will vary depending upon the volume of the tea.

Drymifolia Tea Bioassay

The following study illustrates, but does not limit, the present invention. To test the efficacy of the drymifolia tea containing milled drymifolia leaves having an average particle size of about 11 microns, comparison studies were performed with tea containing hand-crushed drymifolia leaves, LIPITOR®, Haas fruit, and milled leaves of Haas and Nabal having an average particle size of about 11 microns. LIPITOR® is prepared in methanol/buffer to solubilize atorvastatin, the active ingredient in LIPITOR® (40 mg active/600 mg tablet). All test materials were applied to serum culture medium at varying doses.

Tea containing drymifolia leaves having an average particle size of about 11 microns was added to Dulbecco's Modified Eagle Medium (DMEM, Catalogue # 11965, In Vitrogen Corporation, Carlsbad, Calif.) to a final concentration of about 0.1 mg/ml to about 0.5 mg/ml drymifolia leaves via serial dilution (or 0.01% to 0.05% drymifolia leaves). More preferably, the final concentration is about 0.2 mg/ml to about 0.4 mg/ml drymifolia leaves and, most preferably, about 0.4 mg/ml drymifolia leaves. Table 1 shows the components of the culture media which provides all necessary nutrients for cell maintenance including cholesterol synthesis.

TABLE 1

| | Molarity |
|---|---|
| Components of Culture Medium | (mM) |
| INORGANIC SALTS: | |
| Calcium Chloride (CaCl2) (anhyd.) | 1.80 |
| Ferric Nitrate (Fe(NO3)3-9H2O) | 0.000248 |
| Potassium Chloride (KCl) | 5.30 |
| Magnesium Sulfate (MgSO4) | 0.813 |
| Sodium Chloride (NaCl) | 110.34 |
| Sodium Bicarbonate (NaHCO3) | 44.10 |
| Sodium Phosphate (NaH2PO4-H2O) | 0.906 |
| OTHER COMPONENTS: | |
| D-Glucose | 25.00 |
| Phenol red | 0.0346 |
| AMINO ACIDS: | |
| L-Arginine-HCl | 0.398 |
| L-Cystine 2HCl | 0.200 |
| L-Glutamine | 4.00 |
| Glycine | 0.399 |
| L-Histidine HCl—H2O | 0.20 |
| L-Isoleucine | 0.802 |
| L-Leucine | 0.802 |
| L-Lysine-HCl | 0.798 |
| L-Methionine | 0.201 |
| L-Phenylalanine | 0.400 |
| L-Serine | 0.400 |
| L-Threonine | 0.078 |
| L-Tryptophan | 0.078 |
| L-Tyrosine 2Na 2H20 | 0.398 |
| L-Valine | 0.803 |
| VITAMINS: | |
| D-Ca pantothenate | 0.0083 |
| Choline Chloride | 0.0285 |
| Folic Acid | 0.00906 |
| i-Inositol | 0.04 |
| Niacinamide | 0.0328 |
| Pyridoxine HCl | 0.0196 |
| Riboflavin | 0.00106 |
| Thiamine HCl | 0.0118 |

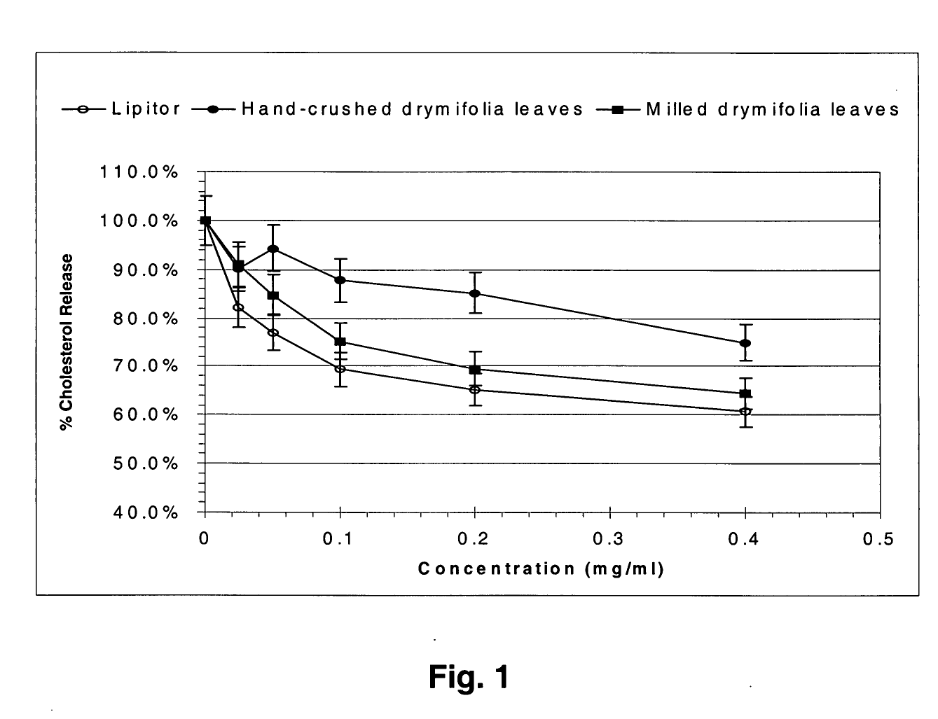

Amounts of secreted cholesterol and cholesteryl ester were measured from acetate fed Hep culture media using a fluorescent indicator, AMPLEX RED. As shown in FIG. 1, it is apparent that the effect of milled drymifolia leaves is comparable to that of LIPITOR®. The results also show that milled drymifolia leaves with an average particle size of about 11 microns had a more favorable result than that of hand-crushed drymifolia leaves when water extracts are made from each. The response of Heps also indicated little or no toxicity associated with the doses treated when cell survival post treatment was assessed via MTT (3-[4,5-dimethylthiazol-2yl]-2,5-diphenyltetrazolium bromide) (Sigma, St. Louis, Mo.) reduction assay.

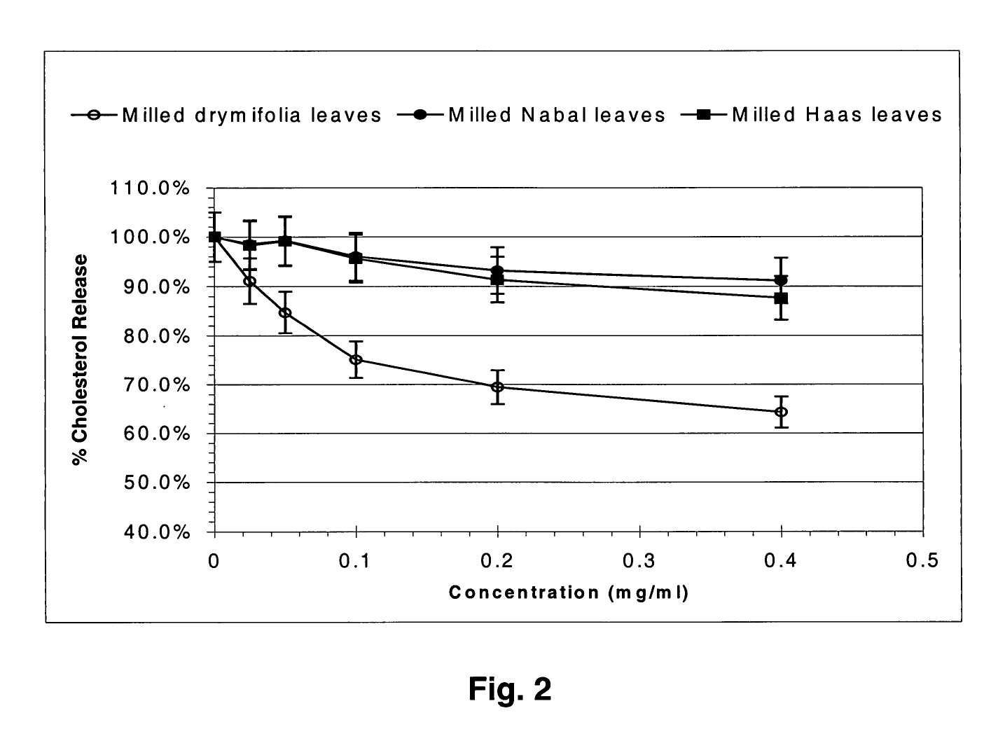

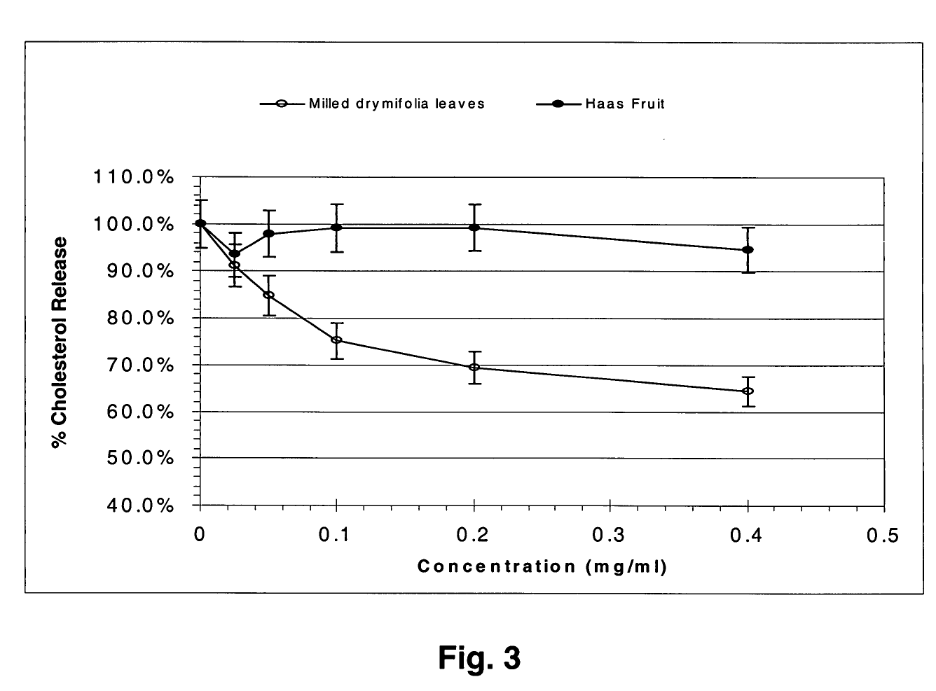

FIG. 2 shows the effect of milled drymifolia leaves to milled leaves of Haas and Nabal. FIG. 2 shows that drymifolia leaves have a more favorable effect in decreasing cholesterol synthesis/secretion than that of Haas or Nabal. As shown in FIG. 3, when the cholesterol inhibitory effect of Haas fruit is assessed in comparison to that of the milled drymifolia leaves, the milled leaves again exerted much more favorable response than the fruit.

Drymifolia Leaf Extract

In another embodiment, drymifolia leaf extract is prepared from dehydrated drymifolia leaves. The extract can be prepared following the milling procedure outlined above where the average particle size of the dehydrated and milled leaves is about 11 microns. In a preferred embodiment, the extract is prepared according to the following procedures. Dehydrated drymifolia leaves are milled using a 60 mesh screen (0.25 mm pore size) or a 16 mesh screen (1 mm pore size) to provide an average particle size of about 250 microns to about 1000 microns, respectively. Preferably, a 60 mesh screen is used to obtain a particle size of about 250 microns. Once the drymifolia leaves are milled and dehydrated, an extract is prepared by slurrying the drymifolia leaves in hot water for about 30 minutes. It is preferable to use a ratio of 10 parts of hot water for each part of drymifolia leaves. Preferably, the water temperature is about 70° C. to about 96° C. More preferably, the water temperature is about 70° C. The hot water extraction continues for 30 minutes with constant stirring. The water and leaves are then cooled to about 45° C. to about 55° C. and, most preferably to about 50° C. The extract is then separated from the cake (also called marc) by screening with an 80 mesh (0.18 mm pore size) Reitz Screw Press (Bepex, Minnesota, Minn.). The cake is squeezed to remove all the extract and thus increase the efficiency of extracting liquid. The liquid extract with soluble solids (which are water soluble compounds only) is concentrated such that the water is evaporated under vacuum to reach a concentration of about 15% soluble solids. The water and leaf mixture is then screen pressed and liquid extracts are allowed to cool to room temperature for fine insoluble solids to settle down in the container. Sediments are screened out through a 200 mesh filter prior to Turba-Film concentration. The soluble solids are then concentrated and spray dried in a chamber where the inlet hot air temperature is about 166° C. (330° F.) and outlet temperature is about 93° C. (200° F.). Table 2 shows data from dehydrated drymifolia leaf lots that have undergone the aforementioned extraction and concentration process. The numerals 3192, 3193, 3194, and 3205 denote different lots of dehydrated drymifolia leaves.

TABLE 2
Extraction Process

| 1. Dehydrate drymifolia leaves, kgs. | | | | 14.0 |
|---|---|---|---|---|
| 2. Tap water, kgs. | | | | 140.0 |
| 3. drymifolia leaf extract after screening, kgs. | | | | 109.2 |
| 4. drymifolia leaf extract refractometer solids, % | | | | 1.8 |
| 5. drymifolia leaf extract pH | | | | 6.5 |
| 6. Wet Cake, kgs. | | | | 45.8 |
| 7. Screw press (S/C) Liquid Extract, kgs. | | | | 22.0 |
| 8. S/C Liquid Extract ref. Solids, % | | | | 1.9 |
| 9. S/C Spent Cake, kgs. | | | | 23.1 |
| 10. S/C Spent Cake moisture, % | | | | 54.3 |
| 11. Turba-Film Extract, kgs. | | | | 15.9 |
| 12. Extract final ref. Solids, % | | | | 16.1 |
| 13. Extract Solids by LOD, % | | | | ? |
| 14. Spray Drying | 3192 | 3193 | 3194 | 3204 |
| Starting Conc., g | 4765 | 4820 | 10400 | 10860 |
| SD Powder, g | 502 | 470 | 1262 | 1144 |
| SD Brushdown, g | 110 | 160 | 56 | 414 |
| Percent Recovery | 88.4 | 90.1 | 81.8 | 97.6 |
| Ratio of Feedstock to | 5.23 | 6 | 4.93 | 4.49 |
| Extract | | | | |

The preferred extraction process outlined above provides a water soluble fraction of drymifolia leaves. Bioassay studies showed that this fraction performs better than the ethanol fraction. One gram of dehydrated and milled drymifolia leaves was extracted with 15 ml of water. Another gram of dehydrated and milled drymifolia leaves was extracted with 15 ml of ethanol. Extractions were carried out by sonication under ambient temperature for 30 minutes. The liquid extracts (fractions) were filtered out from the plant material residue. Solvents were evaporated under nitrogen at 50° C. to obtain dry extracts. These dry extracts (with registered weights) were then diluted to test concentrations and used to treat Hep G2 hepatoma cells to study cholesterol release. Table 3 shows the results of a study comparing cholesterol release of Hep G2 cells treated with a water soluble fraction of drymifolia leaves and cells treated with an ethanol extract of drymifolia leaves. The fraction that shows the greatest cholesterol decrease was the water soluble fraction.

TABLE 3

| | | |
|---|---|---|
| Conc. | | |
| (ug/ml) | Mean | % control |

Water soluble Fraction

| | | | | | | |
|---|---|---|---|---|---|---|
| 33 | 65775 | 63187 | 63468 | 68147 | 65144.3 | 103.95% |
| 100 | 56688 | 55878 | 54234 | 56926 | 55931.5 | 89.25% |

Ethanol soluble Fraction

| | | | | | | |
|---|---|---|---|---|---|---|
| 33 | 63884 | 62268 | 63223 | 67481 | 64214 | 102.46% |
| 100 | 60652 | 68195 | 63855 | 63035 | 63934.3 | 102.01% |

Drymifolia Leaf Extract Bioassay

The following study illustrates, but does not limit, the present invention. To measure cholesterol secretion by hepatoma cells following treatment by drymifolia leaf extract prepared in accordance with the present invention, a bioassay was performed. Samples for the bioassay were prepared by weighing out 100 mg of the spray dried powders of drymifolia leaf extracts. A 50 mg/ml total extract of the sample was then prepared by sequential addition of DMSO:ethanol:water in a ratio of 5:3:2. For example, for 100 mg of powder, 1 ml DMSO, 0.6 ml ethanol, and 0.4 ml water would be used. To prepare dilutions of sample for testing, the samples were diluted from the stock concentration of 50 mg/ml to test concentrations (i.e. 400, 200, 100 μg/ml) in tissue culture media. For the 400 μg/ml concentration, 8 μl of stock solution added to 992 μl tissue culture media would yield 400 μg/ml. From there, serial two fold dilutions would result in the remaining test concentration. Hep G2 hepatoma cells (ATCC, Manassas, Va.) were plated at 2×10⁵/well in standard 24 well tissue culture plates. Following plating, the cells were incubated overnight. The next day, the media in the wells was aspirated and replaced with fresh media containing the diluted sample. Stock solutions of the samples were prepared as total extracts at 50 mg/ml. The samples were then diluted in tissue culture media just prior to adding to the cells. The cells were again incubated overnight with the samples. Then, the supernatant fluids were tested for the presence of cholesterol using the AMPLEX RED cholesterol kit (Molecular Probes, Eugene, Oreg.). The kit measures total cholesterol in the supernatants following enzymatic conversion of any cholesterol esters to free cholesterol using cholesterol esterase. The effect of the samples is calculated by dividing the mean relative fluorescence derived from the test samples by the mean relative fluorescence of untreated controls.

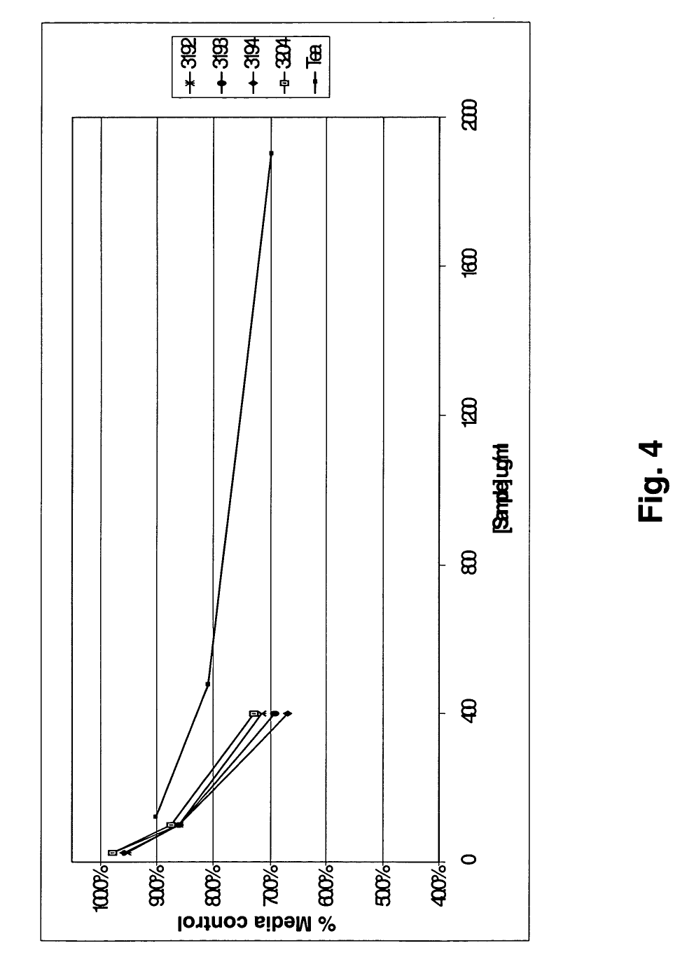

FIG. 4 shows that Hep G2 cells treated with a drymifolia leaf extract loweers cholesterol by about 30% at a dose of 400 ug/ml. The numerals, 3192, 3193, 3192, and 3204, represent different lots of drymifolia leaf extracts prepared for the bioassay.

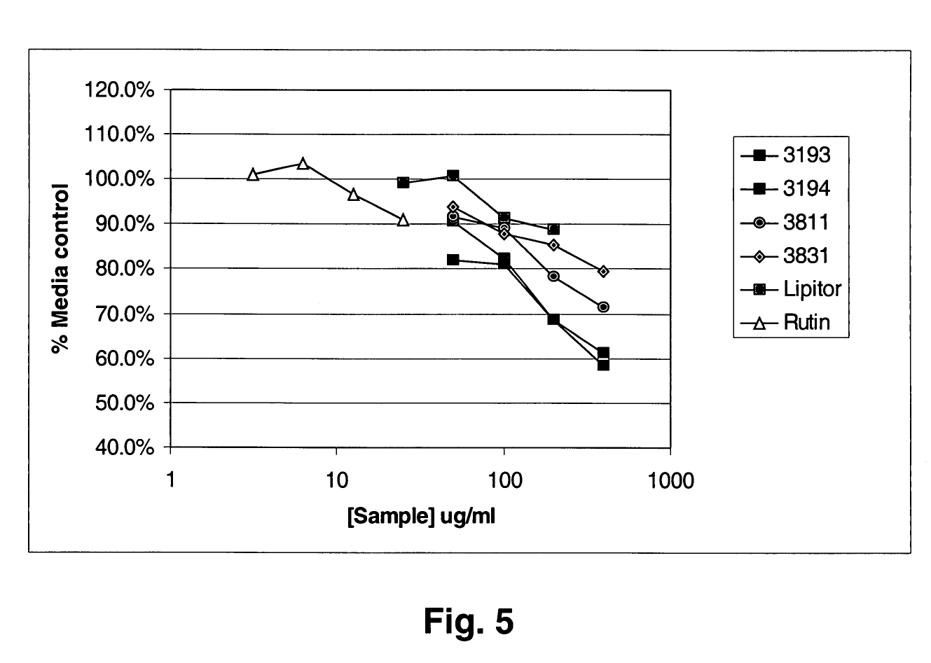

FIG. 5 and Table 4 show that drymifolia leaf extract samples 3193, 3194, 3811, and 3831 on cholesterol release by Hep G2 cells in comparison with other known cholesterol lowering agents: LIPITOR® and pure rutin. LIPITOR® and pure rutin samples were prepared in the same manner as the drymifolia leaf extract in the bioassay. The drymifolia leaf extract samples performed as well as or better than LIPITOR®. The study also suggests that the active(s) primarily responsible for the cholesterol lowering acitivty of drymifolian extract is an agent other than rutin. Pure rutin in the amount equivalent to the amount found in the lots 3193, 3194, 3811, and 3831 was tested. More specifically, the amount of rutin found in the lots was 3.44 mg/g, 4.36 mg/g, 4.42 mg/g and 4.13 mg/g, respectively. At the highest concentration of the drymifolia leaf extract samples tested (which was 400 ug/ml), there could be up to 25 ug/ml of rutin. Thus, the amount of pure rutin used in the bioassay was 3.125 to 25 ug/ml. The extract performed better than pure rutin at a levels equivalent to that present in 400 ug/ml of drymifolian extract.

TABLE 4

| | Conc. | | | | | |
|---|---|---|---|---|---|---|
| Material | (μg/ml) | | | Mean | STD | % ctrl |
| DLC 3193 | 400 | 11474 | 11040 | 11257 | 306.9 | 61.0% |
| | 200 | 12371 | 12963 | 12667 | 418.6 | 68.7% |
| | 100 | 15603 | 14725 | 15164 | 620.8 | 82.2% |
| | 50 | 16484 | 16963 | 16723.5 | 338.7 | 90.6% |
| DLC 3194 | 400 | 11262 | 10249 | 10755.5 | 716.3 | 58.3% |
| | 200 | 12300 | 13065 | 12682.5 | 540.9 | 68.7% |
| | 100 | 15179 | 14755 | 14967 | 299.8 | 81.1% |
| | 50 | 15486 | 14773 | 15129.5 | 504.2 | 82.0% |
| LIPITOR ® | 200 | 15895 | 16861 | 16378 | 683.1 | 88.8% |
| | 100 | 15488 | 18255 | 16871.5 | 1956.6 | 91.5% |
| | 50 | 18207 | 18975 | 18591 | 543.1 | 100.8% |
| | 25 | 18240 | 18377 | 18308.5 | 96.9 | 99.2% |
| Pure Rutin | 25 | 17275 | 16360 | 16817.5 | 647.0 | 91.2% |
| | 12.5 | 18583 | 17087 | 17835 | 1057.8 | 96.7% |
| | 6.25 | 18745 | 19393 | 19069 | 458.2 | 103.4% |
| | 3.125 | 18985 | 18234 | 18609.5 | 531.0 | 100.9% |

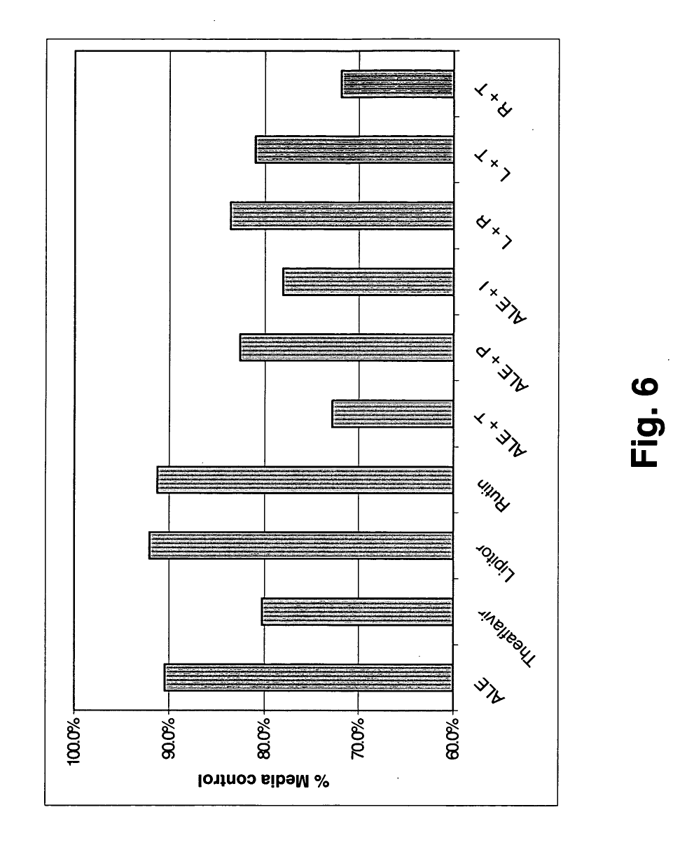

The aforementioned bioassay study was also conducted using drymifolia leaf extract combined with known cholesterol lowering agents such as LIPITOR®, pure rutin, and theaflavin. FIG. 6 shows the results this biosassay. “ALE” denotes drymifolia leaf extract; “T” denotes theaflavin; “L” denotes LIPITOR®; and “R” denotes pure rutin. The concentrations of the tested samples were as follows: 100 mg/ml of ALE; 100 mg/ml of theaflavin; 100 mg/ml of LIPITOR®; and 25 mg/ml of pure rutin. ALE and L showed synergy in lowering cholesterol as did ALE and T. Accordingly, the present invention contemplates the combination of drymifolia leaf extract with other cholesterol lowering agents, including but not limited to, statins, rutin, catechins, theaflavin, krill oil and sterols.

The daily dose of drymifolia leaf extract is at least 200 mg and, more preferably, at least 300 mg and, even more preferably, at least 500 mg. The daily dose is even more preferably 200 mg to about 1000 mg and, most preferably, about 500 mg. The daily dosage can be administered in one composition or in multiple compositions.

The drymifolia leaf extract can be further processed into dietary supplement compositions in the form of, for example, tablets or capsules prepared by conventional means with pharmaceutically or neutraceutically acceptable diluents, carriers, or excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The dietary supplement compositions can optionally contain phytochemicals, vitamins, minerals and flavoring. The tablets may be coated by methods well known in the art. The extract may also be incorporated into foods or beverages. Other oral delivery forms are also contemplated.

The compositions for oral administration may also be formulated to give controlled release of the active compounds. In this regard, the extract of the present invention may be formulated as controlled release powders of discrete micro-particles that can be readily formulated in liquid form. The sustained release powder comprises particles containing an active ingredient and optionally, an excipient with at least one non-toxic polymer.

Depending upon the ingredients and carriers used in a composition, whether it be a food, beverage, or dietary supplement, the composition may include at least about 1% of drymifolia leaf extract. More preferably, the composition includes at least about 20% of drymifolia leaf extract and, most preferably, at least about 40% drymifolia leaf extract.

### EXAMPLE

### Dietary Supplement Tablet

Table 5 is a non-limiting, exemplary, formula for a dietary supplement tablet of the present invention. The tablet in Table 5 is taken twice a day. Those of ordinary skill in the art will appreciate that the total tablet weight of the tablet can vary depending on the type of carriers or excipients used. Additionally, the amount of extract will depend on the number of supplements used to obtain the effective daily dosages.

TABLE 5

| Ingredients | Approx. weight (mg) |
|---|---|
| 1. Microcrystalline Cellulose, Silicified | 47.5 |
| 2. Drymifolia Leaf Extract | 250 |
| 3. Maltodextrin | 19 |
| 4. Silicon Dioxide, NF Fine Powder | 1.2 |
| 5. Sodium Caboxymethylcellulose | 5 |
| 6. Stearic Acid, powder, vegetable | 8 |

The supplement can be prepared by passing ingredients 1 to 5 in Table 5 through a SWECO separator and blended for about 15 minutes. Stearic acid is then passed through a SWECO separator and blended for about 5 minutes. In both instances, the SWECO separator is equipped with a 20 mesh screen directly into a P.K. 50 blender. The combination of ingredients is discharged from the separator into supersacks, totes, or containers, and then compressed and punched by means known to those of ordinary skill in the art to form the tablets.

While the above describes what are presently believed to be the preferred embodiments of the invention, those skilled in the art will realize that changes and modifications may be made thereto without departing from the spirit of the invention. It is intended to claim all such changes and modifications that fall within the true scope of the invention.

1. A composition for lowering cholesterol levels in a mammal comprising a Persea americana var. drymifolia leaf extract.

2. The composition of claim 1 wherein the composition comprises about 200 mg to about 1000 mg of a Persea americana var. drymifolia leaf extract.

3. The composition of claim 1 wherein the composition comprises at least about 200 mg of a Persea americana var. drymifolia leaf extract.

4. The composition of claim 1 wherein the composition comprises a Persea americana var. drymifolia leaf extract in an amount equivalent to the amount of water soluble solids present in 4 drymifolia leaves.

5. The composition of claim 1 wherein the composition comprises at least about 20% of a Persea americana var. drymifolia leaf extract by total weight of the composition.

6. The composition of claim 1 wherein the Persea americana var. drymifolia leaf extract comprises a water-soluble fraction.

7. The composition of claim 1 wherein the composition is selected from the group consisting of: a dietary supplement, a food, and a beverage.

8. The composition of claim 1 further comprising a second cholesterol lowering agent selected from the group consisting of: statins, sterols, krill oil, catechins, and theaflavins.

9. The composition of claim 1 wherein the extract is obtained by the process of:
(a) dehydrating drymifolia leaves;
(b) milling the leaves;
(c) steeping the leaves in 70° C. water;
(d) obtaining a water soluble fraction; and
(e) concentrating the water soluble fraction to about 15% soluble solids.

10. A method of lowering cholesterol levels in a mammal comprising administering a composition comprising a Persea americana var. drymifolia leaf extract.

11. The method of claim 10 wherein the composition is selected from the group consisting of: a dietary supplement, a beverage, and a food.

12. The method of claim 10 wherein the administered amount of the Persea americana var. drymifolia leaf extract is at least about 200 mg per day.

13. The method of claim 10 wherein the administered amount of the Persea americana var. drymifolia leaf extract is at least about 300 mg per day.

14. The method of claim 10 wherein the administered amount of the Persea americana var. drymifolia leaf extract is at least about 500 mg per day.

15. The method of claim 10 wherein the administered amount of the Persea americana var. drymifolia leaf extract is equivalent to the amount of water soluble solids present in 4 drymifolia leaves.

16. The method of claim 10 wherein the composition further comprises a second cholesterol lowering agent.

17. The method of claim 10 wherein the composition further comprises a second cholesterol lowering agent selected from the group consisting of: statins, sterols, krill oil, and catechins, theaflavins.

18. The method of claim 10 wherein the composition is substantially free of rutin.

19. The method of claim 10 wherein the Persea americana var. drymifolia leaf extract comprises a water soluble fraction.

20. A dietary supplement composition for lowering cholesterol levels in mammals comprising a Persea americana var. drymifolia leaf extract and a carrier wherein the extract consists essentially of a water soluble fraction.
